(19) United States
(12) Patent Application Publication
(10) Pub. No.: US 2011/0028498 A1
(43) Pub. Date: Feb. 3, 2011

(54) METHOD FOR EVALUATING PATIENTS FOR TREATMENT WITH DRUGS TARGETING RET RECEPTOR TYROSINE KINASE

(76) Inventors: Anderson Joseph Ryan, Cheshire (GB); James Sherwood, Cheshire (GB); Alan Wookey, Cheshire (GB)

Correspondence Address: ASTRAZENECA R&D BOSTON, 35 GATEHOUSE DRIVE, WALTHAM, MA, 02451-1215, US

(21) Appl. No.: 12/438,306

(22) PCT Filed: Sep. 6, 2007
(86) PCT No.: PCT/GB07/03335

§ 371 Date: Oct. 13, 2009

Related U.S. Application Data
(60) Provisional application No. 60/842,766, filed on Sep. 7, 2006.

(51) Int. Cl. A61K 31/517; C12Q 1/68; C07H 21/04; A61P 35/00

(52) U.S. Cl. 514/266.21; 435/6; 536/24.33

(57) ABSTRACT

The present invention provides a method of selection of a patient, who is a candidate for treatment with a RET drug, whereby to predict an increased likelihood of response to a RET drug. The invention provides a method for determining the sequence of RET. The method provides ARMS primers optimised for determining the sequence of RET. The invention also provides a diagnostic kit, comprising an ARMS primer.

The present invention relates to a method of selection of a patient, who is a candidate for treatment with a RET drug, whereby to predict an increased likelihood of response to a RET drug. The invention provides a method for determining the sequence of RET. The method provides ARMS primers optimized for determining the sequence of RET. The invention also provides a diagnostic kit, comprising an ARMS primer.

The phosphorylation of proteins on tyrosine residues is a key element of signal transduction within cells. Enzymes capable of catalysing such reactions are termed tyrosine kinases. A number of transmembrane receptors contain domains with tyrosine kinase activity and are classified as receptor tyrosine kinases (RTKs). RTKs transduce extracellular signals for processes as diverse as cell growth, differentiation, survival and programmed cell death. In response to binding of extracellular ligands, RTKs typically dimerise, leading to autophosphorylation and intracellular signal transduction through effectors that recognise and interact with the phosphorylated form of the RTK. There are several members of this family of RTKs, one of which is the RET proto-oncogene which encodes the 120 kDa protein RET (Rearranged during Transfection). RET is a receptor for growth factors of the glial-derived neurotrophic factor (GDNF) family. Two ligands for RET have been identified; GDNF and neuturin (NTN). RET is activated when its ligand binds a co-receptor and the complex then interacts with RET (Eng, 1999 Journal Clinical Oncology: 17(1) 380-393).

Activation causes RET to become phosphorylated on tyrosine residues, leading to transduction of signals for cell growth and differentiation through the RAS-RAF and the PI3 kinase pathways and possibly additional routes.

Point mutations that activate RET are known to cause three related, dominantly inherited cancer syndromes; multiple endocrine neoplasia type 2A and 2B (MEN2A and MEN2B) and familial medullary thyroid carcinoma (FMTC) (Santoro et al. 2004 Endocrinology: 145, 5448-5451)

In nearly all MEN2A cases and some FTMC cases there are substitutions of cysteines in the extracellular, juxtamembrane cysteine-rich domain, whereas 95% of MEN2B cases are the result of a single point mutation at codon 918 in the kinase domain (M918T). Codon 918 is thought be located in the substrate recognition pocket of the catalytic core. Mutation at this site is thought to alter the structure of the activation loop of the RET catalytic domain, thereby constitutively activating RET. The M918T mutation is also found in sporadic medullary carcinomas, in which it correlates with an aggressive disease phenotype. In vitro studies have shown that the mutation affects substrate specificity, such that RET recognises and phosphorylates substrates preferred by non-receptor tyrosine kinases such as c-src and c-abl (Eng et al. 1996 JAMA: 276, 1575-1579; Ponder et al. 1999 Cancer Research: 59, 1736-1741; Schilling et al. 2001 International Journal of Cancer: 95, 62-66; Santoro et al. 1995 Science: 267, 381-383; Zhou et al. 1995 Nature: 273, 536-539).

As mutations in the RET gene have been identified in the majority of MEN2 families, molecular diagnostic testing is possible, and can be useful to confirm a clinical diagnosis. Testing for RET mutations can be performed using polymerase chain reaction-based protocols; wherein target exonic sequences are amplified for direct sequencing or restriction endonuclease digestion (Zhong et al. 2006 Clinica Chimica Acta: 364, 205-208).

Another member of the family of RTKs is vascular endothelial growth factor receptor 2 (VEGFR2 (the kinase insert domain-containing receptor, KDR (also referred to as Flk-1))). VEGFR2 is a receptor for vascular endothelial growth factor (VEGF). VEGF is believed to be an important stimulator of both normal and disease-related angiogenesis (Jakeman, et al. 1993 Endocrinology: 133, 848-859; Kolch, et al. 1995 Breast Cancer Research and Treatment: 36, 139-155) and vascular permeability (Connolly, et al. 1989 J. Biol. Chem.: 264, 20017-20024). Antagonism of VEGF action by sequestration of VEGF with antibody can result in inhibition of tumour growth (Kim, et al. 1993 Nature: 362, 841-844). Heterozygous disruption of the VEGF gene resulted in fatal deficiencies in vascularisation (Carmeliet, et al. 1996 Nature 380:435-439; Ferrara, et al. 1996 Nature 380:439-442).

Binding of VEGF to VEGFR2 leads to receptor dimerisation, causing VEGFR2 autophosphorylation of specific intracellular tyrosine residues. Autophosphorylation increases the catalytic activity of the tyrosine kinase and provides potential docking sites for cytoplasmic signal transduction molecules such as phospholipase C-γ. These protein interactions mediate the intracellular signaling necessary to induce cellular response to VEGFR2, for example endothelial cell proliferation, survival and migration (Ryan et al. 2005 British Journal Cancer: 92(Suppl.1) S6-S13).

Recognition of the key role of VEGF-mediated VEGFR2 signalling in pathological angiogenesis has led to the development of various selective approaches to inhibit VEGFR2 activation. These include small molecule ATP-competitive tyrosine kinase inhibitors, which in preventing ATP binding preclude autophosphorylation and subsequent intracellular signal transduction (Ryan, 2005).

Quinazoline derivatives which are inhibitors of VEGF receptor tyrosine kinase are described in International Patent Applications Publication Nos. WO 98/13354 and WO 01/32651. In WO 98/13354 and WO 01/32651 compounds are described which possess activity against VEGF receptor tyrosine kinase whilst possessing some activity against epidermal growth factor receptor (EGFR) tyrosine kinase.

It has been disclosed (Wedge et al. 2002 Cancer Research: 62, 4645-4655) that the compound 4-(4-bromo-2-fluoroanilino)-6-methoxy-7-(1-methylpiperidin-4-ylmethoxy)quinazoline is a VEGFR2 tyrosine kinase inhibitor. This compound is also known as Zactima™ (registered trade mark), by the generic name vandetanib and by way of the code number ZD6474. The compound is identified hereinafter as vandetanib.

Vandetanib was developed as a potent and reversible inhibitor of ATP-binding to VEGFR2 tyrosine kinase. In addition, vandetanib also inhibits EGFR tyrosine kinase activity. The EGFR signalling pathway is also key to cancer progression, where aberrant EGFR activity increases tumour cell proliferation, survival and invasiveness as well as the overexpression of VEGF. Inhibition of EGFR signalling has been shown to induce selective apoptosis in tumour endothelial cells.

In 2002 it was reported that vandetanib had demonstrated potent inhibition of ligand-dependent RET tyrosine kinase activity thereby inhibiting the signalling and transforming capacity of RET. Furthermore, vandetanib demonstrated a strong growth-inhibitory effect on RET-dependent thyroid tumour cell growth in vitro (Carlomagno et al. 2002 Cancer Research: 62, 7284-7290). Vandetanib inhibited the majority of mutated, activated forms of RET and also the wild type receptor. Therefore in addition to inhibition of VEGFR2 and EGFR tyrosine kinase, it is thought that inhibition of RET tyrosine kinase by vandetanib may contribute additional antitumour effects in treating tumours with mutations in the RET gene which lead to RET-dependent tumour cell growth (Ryan, 2005).

The present invention permits the selection of a patient, who is a candidate for treatment with a RET drug, in order to predict an increased likelihood of response to a RET drug. As mutations that constitutively activate RET are known to lead to several RET-signaling dependent cancer syndromes, determination of these mutations in a patient can be used to assess the suitability of a patient for treatment with a RET drug.

According to one aspect of the invention there is provided a method for predicting the likelihood that a patient who is a candidate for treatment with a RET drug will respond to said treatment, comprising determining the sequence of RET in a sample obtained from the patient at the following position as defined in SEQ ID NO: 1: position 105, is not thymine. In one embodiment, the method comprises determining whether the sequence of RET in a sample obtained from the patient at position 105, as defined in SEQ ID NO:1, is not thymine, whereby to predict an increased likelihood of response to the RET drug. In one embodiment, the method comprises determining the sequence of RET in a sample obtained from the patient at the following position as defined in SEQ ID NO: 1: position 105, is cytosine. In one embodiment, the method comprises determining whether the sequence of RET in a sample obtained from the patient at position 105, as defined in SEQ ID NO:1, is cytosine, whereby to predict an increased likelihood of response to the RET drug.

According to another aspect of the invention there is provided a method for predicting the likelihood that a patient who is a candidate for treatment with a RET drug will respond to said treatment, comprising determining the sequence of RET in a sample obtained from the patient at the following position as defined in SEQ ID NO: 2: position 918, is not methionine.

In one embodiment, the method comprises determining whether the sequence of RET in a sample obtained from the patient at position 918, as defined in SEQ ID NO:2, is not methionine, whereby to predict an increased likelihood of response to the RET drug. In one embodiment, the method comprises determining the sequence of RET in a sample obtained from the patient at the following position as defined in SEQ ID NO: 2: position 918, is threonine. In one embodiment, the method comprises determining whether the sequence of RET in a sample obtained from the patient at position 918, as defined in SEQ ID NO:2, is threonine, whereby to predict an increased likelihood of response to the RET drug.

In one embodiment, there is provided a method for predicting the likelihood that a patient who is a candidate for treatment with a RET drug will respond to said treatment, comprising determining whether the sequence of RET in a sample obtained from the patient at the following position as defined in SEQ ID NO:1: position 105, is cytosine, or at the following position as defined in SEQ ID NO:2: position 918, is threonine, whereby to predict an increased likelihood of response to a RET drug.

In one embodiment the present invention is particularly suitable for use in predicting the response of a patient, who is a candidate for treatment with a RET drug, to a RET drug, in patients with a tumour which is dependent alone, or in part, on RET. In one embodiment the present invention is particularly suitable for use in predicting the response to a RET drug, in patients with a tumour which is dependent alone, or in part, on mutant RET. Such tumours include, for example, thyroid carcinomas. In another embodiment the present invention is particularly suitable for use in predicting the response to a RET drug, in patients with a tumour selected from medullary thyroid carcinoma, an adrenal gland tumour (such as phaeochromocytoma) lung cancer (especially small cell lung cancer), papillary thyroid carcinoma, mesothelioma and colorectal cancer. In another embodiment the present invention is particularly suitable for use in predicting the response to a RET drug, in patients with a tumour selected from medullary thyroid carcinoma, an adrenal gland tumour (such as phaeochromocytoma) and lung cancer (especially small cell lung cancer).

In another embodiment the present invention is particularly suitable for use in predicting the likelihood that a patient who is a candidate for treatment with a RET drug will respond to said treatment, in patients with a tumour which is dependent alone, or in part, on RET. In one embodiment the present invention is particularly suitable for use in predicting the likelihood that a patient who is a candidate for treatment with a RET drug will respond to said treatment, in patients with a tumour which is dependent alone, or in part, on mutant RET. Such tumours include, for example, thyroid carcinomas. In another embodiment the present invention is particularly suitable for use in predicting the likelihood that a patient who is a candidate for treatment with a RET drug will respond to said treatment, in patients with a tumour selected from medullary thyroid carcinoma, an adrenal gland tumour (such as phaeochromocytoma) lung cancer (especially small cell lung cancer), papillary thyroid carcinoma, mesothelioma and colorectal cancer. In another embodiment the present invention is particularly suitable for use in predicting the likelihood that a patient who is a candidate for treatment with a RET drug will respond to said treatment, in patients with a tumour selected from medullary thyroid carcinoma, an adrenal gland tumour (such as phaeochromocytoma) and lung cancer (especially small cell lung cancer).

In one embodiment of the invention there is provided a method as described hereinabove wherein the method for detecting a nucleic acid mutation in RET and thereby determining the sequence of RET, is selected from sequencing, WAVE analysis, amplification refractory mutation system (ARMS) and restriction fragment length polymorphism (RFLP). ARMS is described in European Patent, Publication No. 0332435, the contents of which are incorporated herein by reference, which discloses and claims a method for the selective amplification of template sequences which differ by as little as one base, which method is now commonly referred to as ARMS. RFLP is described by Zhong (Zhong et al: 2006 Clinica Chimica Acta: 364, 205-208). In one embodiment of the invention there is provided a method as described hereinabove wherein the method for determining the sequence of RET in a sample obtained from a patient is selected from any one of amplification refractory mutation system, restriction fragment length polymorphism or WAVE analysis. In one embodiment of the invention there is provided a method as described hereinabove wherein the method for determining the sequence of RET in a sample obtained from a patient is the amplification refractory mutation system. In one embodiment ARMS may comprise use of an agarose gel, sequencing gel or real-time PCR. In one embodiment ARMS comprises use of real-time PCR. The ARMS assay may be multiplexed with a second PCR reaction that detects the presence of DNA in the reaction, thereby indicating successful PCR. TaqMan™ technology may be used to detect the PCR products of both reactions using TaqMan™ probes labelled with different fluorescent tags. The advantages of using ARMS rather than sequencing or RFLP to detect mutations are that ARMS is a quicker single step assay, less processing and data analysis is required, and ARMS can detect a mutation in a sample against a background of wild type polynucleotide.

In one embodiment of the invention there is provided a method of determining the sequence of RET in a sample obtained from a patient comprising use of an ARMS mutant forward primer capable of recognising the sequence of RET at position 105 as shown in SEQ ID NO:1. In one embodiment of the invention there is provided a method of determining the sequence of RET in a sample obtained from a patient comprising use of an ARMS mutant forward primer and an ARMS reverse primer optimized to amplify the region of a RET sequence comprising position 105 as shown in SEQ ID NO:1. The skilled person would understand that “optimized to amplify” comprises determining the most appropriate length and position of the forward primer and reverse primer. In one embodiment the ARMS mutant forward primer and the ARMS reverse primer are optimized to amplify a region of less than 500 bases. In one embodiment the ARMS mutant forward primer and the ARMS reverse primer are optimized to amplify a region of less than 250 bases. In one embodiment the ARMS mutant forward primer and the ARMS reverse primer are optimized to amplify a region of less than 200 bases. In one embodiment the ARMS mutant forward primer and the ARMS reverse primer are optimized to amplify a region of greater than 100 bases.

In one embodiment the ARMS mutant forward primer is capable of recognising the sequence of RET at position 105 as defined in SEQ ID NO: 1. In one embodiment the ARMS mutant forward primer comprises a sequence at least 75%, 80%, 85%, 90%, 95%, 96%, 97%, 98% or 99% identical to the sequence disclosed in SEQ ID NO:3. In another embodiment the ARMS mutant forward primer comprises SEQ ID NO:3. In a further embodiment the ARMS mutant forward primer consists of SEQ ID NO:3.

In one embodiment the ARMS reverse primer comprises a sequence at least 75%, 80%, 85%, 90%, 95%, 96%, 97%, 98% or 99% identical to the sequence disclosed in SEQ ID NO:4. In one embodiment the ARMS reverse primer comprises SEQ ID NO:4. In one embodiment the ARMS reverse primer consists of SEQ ID NO:4.

Locked Nucleic Acid (LNA) oligonucleotides contain a methylene bridge connecting the 2′-oxygen of ribose with the 4′-carbon. This bridge results in a locked 3′-endo conformation, reducing the conformational flexibility of the ribose and increasing the local organisation of the phosphate backbone. Braasch and Corey have reviewed the properties of LNA/DNA hybrids (Braasch and Corey, 2001, Chemistry & Biology 8, 1-7).

Several studies have shown that primers comprising LNAs have improved affinities for complementary DNA sequences. Incorporation of a single LNA base can allow melting temperatures (Tm) to be raised by up to 41° C. when compared to DNA:DNA complexes of the same length and sequence, and can also raise the Tm values by as much as 9.6° C. Braasch and Corey propose that inclusion of LNA bases will have the greatest effect on oligonucleotides shorter than 10 bases.

Implications of the use of LNA for the design of PCR primers have been reviewed (Latorra, Arar and Hurley, 2003, Molecular and Cellular Probes 17, 253-259). It was noted that firm primer design rules had not been established but that optimisation of LNA substitution in PCR primers was complex and depended on number, position and sequence context. Ugozolli et al (Ugozolli, Latorra, Pucket, Arar and Hamby, 2004, Analytical Biochemistry 324, 143-152) described the use of LNA probes to detect SNPs in real-time PCR using the 5′ nuclease assay. Latorra et al (Latorra, Campbell, Wolter and Hurley, 2003, Human Mutation 22, 79-85) synthesised a series of primers containing LNA bases at the 3′ terminus and at positions adjacent to the 3′ terminus for use as allele specific primers. Although priming from mismatched LNA sequences was reduced relative to DNA primers, optimisation of individual reactions was required.

In one embodiment the ARMS mutant forward primer comprises a sequence in which one or more of the standard DNA bases have been substituted with a LNA base. In one embodiment the ARMS mutant forward primer comprises a sequence at least 75%, 80%, 85%, 90%, 95%, 96%, 97%, 98% or 99% identical to the sequence disclosed in SEQ ID NO:9. In another embodiment the ARMS mutant forward primer comprises SEQ ID NO:9. In a further embodiment the ARMS mutant forward primer consists of SEQ ID NO:9.

In one embodiment there is provided an ARMS probe capable of binding to the amplification product resulting from use of an ARMS mutant forward primer and an ARMS reverse primer as described hereinabove in an ARMS assay. In one embodiment the ARMS probe comprises a sequence at least 75%, 80%, 85%, 90%, 95%, 96%, 97%, 98% or 99% identical to the sequence disclosed in SEQ ID NO:5. In another embodiment the ARMS probe comprises SEQ ID NO:5. In a further embodiment the ARMS probe consists of SEQ ID NO:5. In one embodiment the ARMS probe comprises a sequence in which one or more of the standard DNA bases have been substituted with a LNA base. In one embodiment the ARMS probe comprises a sequence at least 75%, 80%, 85%, 90%, 95%, 96%, 97%, 98% or 99% identical to the sequence disclosed in SEQ ID NO:10. In another embodiment the ARMS probe comprises SEQ ID NO:10. In a further embodiment the ARMS probe consists of SEQ ID NO:10. In one embodiment the ARMS probe comprises a Yakima Yellow™ fluorescent tag on the 5′ end. In one embodiment the ARMS probe comprises a BHQ™ quencher on the 3′ end. The skilled person would recognise that the position at which the probe binds in the amplified product (and thus the sequence of the probe is complementary to) is restricted only by the boundaries imposed by the forward and reverse primers which determine the amplified product.

The Control probe is used to confirm that the ARMS assay is working as intended and to confirm that there is DNA in the sample used in the ARMS assay. The skilled person would understand that the Control probe could be targeted to any chosen gene. In one embodiment the Control forward primer comprises a sequence at least 75%, 80%, 85%, 90%, 95%, 96%, 97%, 98% or 99% identical to the sequence disclosed in SEQ ID NO:6. In another embodiment the Control forward primer primer comprises SEQ ID NO:6. In a further embodiment the Control forward primer primer consists of SEQ ID NO:6. In one embodiment the Control reverse primer comprises a sequence at least 75%, 80%, 85%, 90%, 95%, 96%, 97%, 98% or 99% identical to the sequence disclosed in SEQ ID NO:7. In another embodiment the Control reverse primer primer comprises SEQ ID NO:7. In a further embodiment the Control reverse primer primer consists of SEQ ID NO:7. In one embodiment the Control probe comprises a sequence at least 75%, 80%, 85%, 90%, 95%, 96%, 97%, 98% or 99% identical to the sequence disclosed in SEQ ID NO:8. In another embodiment the Control probe comprises SEQ ID NO:8. In a further embodiment the Control probe consists of SEQ ID NO:8. In one embodiment the Control probe comprises a sequence in which one or more of the standard DNA bases have been substituted with a LNA base. In one embodiment the Control probe comprises a sequence at least 75%, 80%, 85%, 90%, 95%, 96%, 97%, 98% or 99% identical to the sequence disclosed in SEQ ID NO:11. In another embodiment the Control probe comprises SEQ ID NO:11. In a further embodiment the Control probe consists of SEQ ID NO:11. In one embodiment the Control probe comprises a Cy™-5 fluorescent tag on the 5′ end. In one embodiment the Control probe comprises a ElleQuencher™ quencher on the 3′ end.

TABLE 1
ARMS Assay Primers and Probes

| | | | | SEQ |
|---|---|---|---|---|
| | | | 3′ | ID |
| Primer | 5′ Mod | Primer Sequence | Mod | NO. |
| ARMS | | CTTTAGTGTCGGATTCCAGTTAAATGGTC | | 3 |
| Mutant | | | | |
| Forward | | | | |
| Primer | | | | |
| ARMS | | T + CGG + ATT + CCA + GT + TAAATGGT + C | | 9 |
| LNA | | | | |
| Mutant | | | | |
| Forward | | | | |
| Primer | | | | |
| ARMS | | TGCAATTCCCTGGCCAAGCTGC | | 4 |
| Reverse | | | | |
| Primer | | | | |
| Short | | | | |
| ARMS | Yakima | CTACACCACGCAAAGTGATGTGTAAGTGT | BHQ ™ | 5 |
| Probe | Yellow ™ | GGGTGTTGCTC | | |
| ARMS | Yakima | TGA + TG + TG + TAAGTGTG + GGTGTTG + CT | BHQ ™ | 10 |
| LNA | Yellow ™ | C | | |
| Probe | | | | |
| Control | | AGGACACCGAGGAAGAGGACTT | | 6 |
| Primer | | | | |
| Forward | | | | |
| Control | | GGAATCACCTTCTGTCTTCATTT | | 7 |
| Primer | | | | |
| Reverse | | | | |
| Control | Cy ™-5 | CCATCTTCTTCCTGCCTGATGAGGGGAAA | ElleQuencher | 8 |
| Probe | | | | |
| Control | Cy ™-5 | CTGC + CT + GA + TGAGGGGAA | ElleQuencher | 11 |
| LNA | | | | |
| Probe | | | | |

The control gene is alantitrypsin. Yakima Yellow and Cy™-5 are fluorescent tags and BHQ™ (Black Hole Quencher™) and ElleQuencher are quenchers.
Emboldened underlined bases indicate mismatch positions. LNA substitution indicated by ‘+’ e.g. +C, +A, +T and +G.

In another aspect of the invention there is provided a method as described hereinabove wherein the method for determining the sequence of RET comprises determining the sequence of cDNA generated by reverse transcription of RET mRNA extracted from archival tumour sections or other clinical material. Extraction of RNA from formalin fixed tissue has been described in Bock et al., 2001 Analytical Biochemistry: 295 116-117, procedures for extraction of RNA from non-fixed tissues, and protocols for generation of cDNA by reverse transcription, PCR amplification and sequencing are described in Sambrook, J. and Russell, D. W., Molecular Cloning: A Laboratory Manual, the third edition, Cold Spring Harbor Laboratory Press, Cold Spring Harbor, N.Y., 2001.

In another aspect of the invention there is provided a method as described hereinabove wherein the method for determining the sequence of RET comprises amplification of individual exons of the RET gene, heteroduplex annealing of individual exons followed by digestion with Cel I (as described in Crepin et al., 2006 Endocrinology: 36, 369-376; and Marsh et al., 2001 Neoplasia: 3, 236-244).

In another aspect, the invention provides a mutant human RET polynucleotide comprising the following nucleic acid base at the following position as defined in SEQ ID NO: 1: a cytosine at position 105, or a fragment thereof comprising at least 20 nucleic acid bases provided that the fragment comprises position 105.

In a further aspect the invention provides a mutant human RET polypeptide comprising the following amino acid residue at the following position as defined in SEQ ID NO: 2: a threonine at position 918, or a fragment thereof comprising at least 10 amino acid residues provided that the fragment comprises position 918.

In another aspect, there is provided a method for determining the sequence of RET in mRNA encoded by a mutant RET gene.

In another aspect of the invention there is provided a method as described herein wherein the method for determining the sequence of RET is selected from, for example, an immunohistochemistry-based assay which may use a slide from a single patient, or a tissue microarray (Mayr et al., 2006 American Journal of Clinical Pathology: 126, 101-109; Zheng et al., 2006 Anticancer Research: 26, 2353-2360) or application of an alternative proteomics methodology, which could comprise lysing cells, digesting the proteins, separating protein fragments on a gel, obtaining the peptide containing the mutated amino acid and analysing the peptide by mass spectrometry.

In another aspect the invention provides an antibody specific for a mutant human RET polypeptide as defined hereinabove.

A further aspect of the invention provides a diagnostic kit, comprising an ARMS mutant forward primer capable of detecting a mutation in RET at position 105, as defined in SEQ ID NO: 1, and optionally an ARMS reverse primer, and optionally instructions for use. In one embodiment of the invention there is provided a diagnostic kit, comprising an ARMS mutant forward primer comprising one or more LNA bases and capable of recognising the sequence of RET at position 105, as defined in SEQ ID NO: 1, and optionally an ARMS reverse primer, and optionally instructions for use. In one embodiment the diagnostic kit may be used in a method of predicting the likelihood that a patient, who is a candidate for treatment with a RET drug, will respond to said treatment. In an alternative embodiment the diagnostic kit may be used in selecting a patient, who is a candidate for treatment with a RET drug, for said treatment. In an alternative embodiment the diagnostic kit may be used to assess the suitability of a patient, who is a candidate for treatment with a RET drug, for said treatment.

A further aspect of the invention provides a diagnostic kit, comprising an antibody specific for a mutant human RET polypeptide as defined hereinabove, and optionally instructions for use. In one embodiment the diagnostic kit may be used in a method of predicting the likelihood that a patient, who is a candidate for treatment with a RET drug, will respond to said treatment. In an alternative embodiment the diagnostic kit may be used in selecting a patient, who is a candidate for treatment with a RET drug, for said treatment. In an alternative embodiment the diagnostic kit may be used to assess the suitability of a patient, who is a candidate for treatment with a RET drug, for said treatment.

In a further aspect of the invention the ARMS primers and probes as described hereinabove may be used to determine the sequence of RET in a panel of cell lines expressing either the wild type or a mutant RET. Knowledge of whether the cell lines are expressing either wild type or mutant RET could be used in screening programmes to identify novel RET inhibitors with specificity for the mutant RET phenotype or novel inhibitors with activity against the phenotype associated with the wild type receptor. The availability of a panel of cell lines expressing mutant RETs will assist in the definition of the signaling pathways activated through RET and may lead to the identification of additional targets for therapeutic intervention.

In another aspect the invention provides a method of preparing a personalised genomics profile for a patient comprising determining the sequence of RET in a sample obtained from the patient at the following position as defined in SEQ ID NO: 1: position 105, and/or the following position as defined in SEQ ID NO: 2: position 918, and creating a report summarising the data obtained by said analysis.

In a specific embodiment, the method as described hereinabove may be used to assess the pharmacogenetics of a RET drug. Pharmacogenetics is the study of genetic variation that gives rise to differing response to drugs. By determining the sequence of RET in a sample obtained from a patient and analysing the response of the patient to a RET drug, the pharmacogenentics of the RET drug can be elucidated.

In one embodiment the method for predicting the likelihood that a patient who is a candidate for treatment with a RET drug will respond to said treatment, may be used to select a patient, or patient population, with a tumour for treatment with a RET drug.

In one embodiment the method for predicting the likelihood that a patient who is a candidate for treatment with a RET drug will respond to said treatment, may be used to predict the responsiveness of a patient, or patient population, with a tumour to treatment with a RET drug.

The sample obtained from the patient may be any tumour tissue or any biological sample that contains material which originated from the tumour, for example a blood sample containing circulating tumour cells or DNA. In one embodiment the blood sample may be whole blood, plasma, serum or pelleted blood. In one embodiment a tumour sample is a tumour tissue sample. The tumour tissue sample may be a fixed or unfixed sample. In another embodiment the biological sample would have been obtained using a minimally invasive technique to obtain a small sample of tumour, or suspected tumour, from which to determine the RET sequence. In another embodiment the biological sample comprises either a single sample, which may be tested for any of the mutations as described hereinabove, or multiple samples, which may be tested for any of the mutations as described hereinabove.

According to another aspect of the invention there is provided a method of using the results of the methods described above in determining an appropriate dosage of a RET drug. For example, knowledge that a patient is predicted to have an increased likelihood of response to a RET drug, could be used in determining an appropriate dosage of the RET drug. Calculating therapeutic drug dose is a complex task requiring consideration of medicine, pharmacokinetics and pharmacogenetics. The therapeutic drug dose for a given patient will be determined by the attending physician, taking into consideration various factors known to modify the action of drugs including severity and type of disease, body weight, sex, diet, time and route of administration, other medications and other relevant clinical factors. Therapeutically effective dosages may be determined by either in vitro or in vivo methods.

A RET drug is a RET inhibitor. A RET inhibitor is an agent that inhibits the activity of RET. Said agent may be an antibody or a small molecule. In one embodiment a RET inhibitor is a RET tyrosine kinase inhibitor. A RET inhibitor may have activity against other proteins, such as inhibition of the activity of other tyrosine kinases, for example VEGFR2 and/or EGFR. In one embodiment a RET inhibitor also inhibits EGFR tyrosine kinase activity. In one embodiment a RET inhibitor also inhibits VEGFR2 tyrosine kinase activity. In one embodiment a RET inhibitor also inhibits VEGFR2 and EGFR tyrosine kinase activity.

RET, EGFR or VEGFR2 tyrosine kinase inhibitors include vandetanib, cediranib (AZD2171, Recentin™, (Wedge et al., 2005 Cancer Research: 65, 4389-4400)), gefitinib, erlotinib, sunitinib (SU11248, Sutent®, Pfizer), SU14813 (Pfizer), vatalanib (Novartis), sorafenib (BAY43-9006, Nexavar, Bayer), XL-647 (Exelixis), XL-999 (Exelixis), AG-013736 (Pfizer), motesanib (AMG706, Amgen), BIBF1120 (Boehringer), TSU68 (Taiho), GW786034, AEE788 (Novartis), CP-547632 (Pfizer), KRN 951 (Kirin), CHIR258 (Chiron), CEP-7055 (Cephalon), OSI-930 (OSI Pharmaceuticals), ABT-869 (Abbott), E7080 (Eisai), ZK-304709 (Schering), BAY57-9352 (Bayer), L-21649 (Merck), BMS582664 (BMS), XL-880 (Exelixis), XL-184 (Exelixis) or XL-820 (Exelixis).

In one embodiment the RET inhibitor is selected from vandetanib, cediranib, sunitinib, motesanib or an antibody. In one embodiment the RET inhibitor is vandetanib. In one embodiment the RET inhibitor is cediranib. In one embodiment the RET inhibitor is motesanib. In one embodiment the RET inhibitor is sunitinib. In one embodiment the RET inhibitor is an antibody.

An effective amount of a RET drug will depend, for example, upon the therapeutic objectives, the route of administration, and the condition of the patient. Accordingly, it is preferred for the therapist to titer the dosage and modify the route of administration as required to obtain the optimal therapeutic effect. A typical daily or intermittent dosage, such as weekly, fortnightly or monthly, might range from about 0.5 mg to up to 300 mg, 500 mg, 1000 mg or 1200 mg or more, depending on the factors mentioned above.

We contemplate that a RET drug may be used as monotherapy or in combination with other drugs. The present invention is also useful in adjuvant, or as a first-line, therapy.

In one embodiment the method of the present invention additionally comprises administration of a RET drug to a patient selected for, or predicted to respond to treatment with a RET drug according the methods described hereinabove.

In one embodiment of the invention there is provided use of a RET drug in preparation of a medicament for treating a patient, or a patient population, selected for, or predicted to respond to, treatment with a RET drug according the methods described hereinabove.

In one embodiment of the invention there is provided a method of treating a patient, or a patient population, selected for, or predicted to have an increased likelihood of response to a RET drug according to the method as described herein, comprising administering a RET drug to said patient(s).

In one embodiment of the invention there is provided a method of treating a patient who is a candidate for treatment with a RET drug comprising:
(i) determining whether the sequence of RET in a sample obtained from the patient at the following position as defined in SEQ ID NO: 1: position 105, is not thymine; or
(ii) determining whether the sequence of RET in a sample obtained from the patient at the following position as defined in SEQ ID NO: 2: position 918, is not methionine;
   and administering an effective amount of the RET drug.

In one embodiment of the invention there is provided a method of treating a patient who is a candidate for treatment with a RET drug comprising:
(i) determining whether the sequence of RET in a sample obtained from the patient at position 105, as defined in SEQ ID NO: 1, is not thymine; or
(ii) determining whether the sequence of RET in a sample obtained from the patient at position 918, as defined in SEQ ID NO: 2, is not methionine;
   and administering an effective amount of the RET drug.

In one embodiment of the invention there is provided a method of treating a patient who is a candidate for treatment with a RET drug, comprising:
(i) determining whether the sequence of RET in a sample obtained from the patient at the following position as defined in SEQ ID NO: 1: position 105, is cytosine; or
(ii) determining whether the sequence of RET in a sample obtained from the patient at the following position as defined in SEQ ID NO: 2: position 918, is threonine,
   and administering an effective amount of the RET drug.

In one embodiment of the invention there is provided a method of treating a patient who is a candidate for treatment with a RET drug comprising:
(i) determining whether the sequence of RET in a sample obtained from the patient at position 105, as defined in SEQ ID NO: 1, is cytosine; or
(ii) determining whether the sequence of RET in a sample obtained from the patient at position 918, as defined in SEQ ID NO: 2, is threonine;
   and administering an effective amount of the RET drug.

### EXAMPLES

The invention is illustrated by the following non-limiting examples, in which

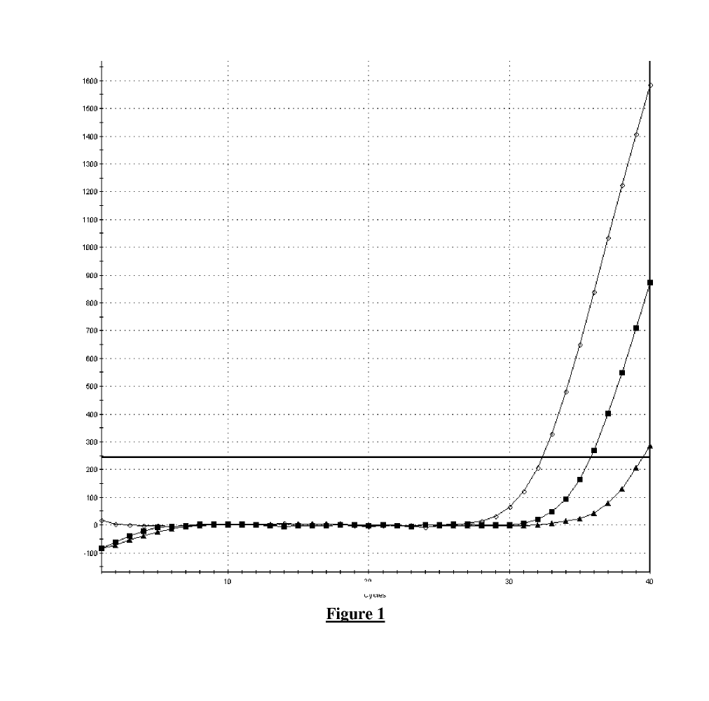

FIG. 1. Shows detection of M918T RET mutation using conventional DNA ARMS primers. The open diamonds show the signal obtained with 1000 copies of mutant DNA, black squares show the signal obtained with 10000 copies of wild type DNA, and black diamonds show the signal obtained with 1000 copies of wild type DNA.

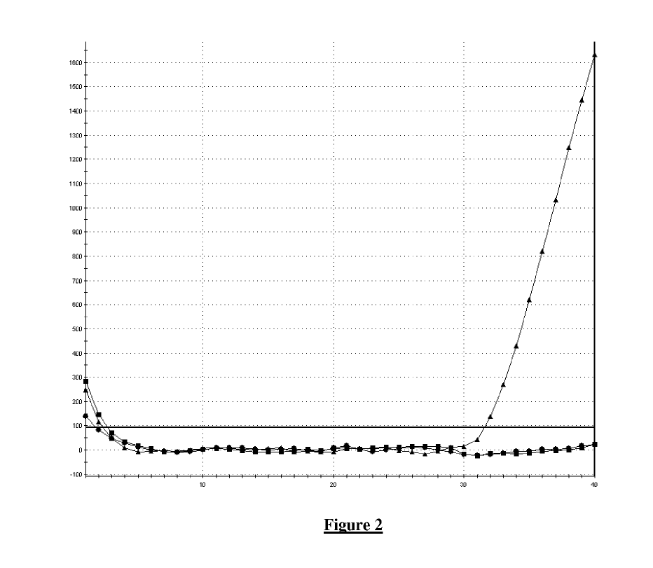

FIG. 2. Shows detection of M918T RET mutation using LNA modified ARMS primers. The black triangles show the signal obtained with 1000 copies of mutant DNA, black squares show the signal obtained with 1000 copies of wild type DNA, and black circles show the signal obtained with 10000 copies of wild type DNA.

General molecular biology techniques are described in “Current Protocols in Molecular Biology Volumes 1-3, edited by F M Asubel, R Brent and R E Kingston; published by John Wiley, 1998 and Sambrook, J. and Russell, D. W., Molecular Cloning: A Laboratory Manual, the third edition, Cold Spring Harbor Laboratory Press, Cold Spring Harbor, N.Y., 2001.

### Example 1

### Identification of Mutations in Sporadic Medullary Thyroid Tumour Sections

Tumour sections were taken from patients at the time of diagnosis or surgery. The sections were formalin fixed and embedded in paraffin wax. The prepared samples were cut into sections, which varied in thickness from 5-20 microns. Regions of section containing tumour were identified by histopathology of a master slide and tumour material was recovered from the relevant area of adjacent slides cut from the same tumour sample, as described by Lynch et al. 2004 New England Journal of Medicine: 350 2129-2139. Other types of tumour sample could include for example, fresh or frozen tissue or circulating tumour cells. Details of techniques using such samples may be found in Sambrook, J. and Russell, D. W., Molecular Cloning: A Laboratory Manual, the third edition, Cold Spring Harbor Laboratory Press, Cold Spring Harbor, N.Y., 2001.

### Example 2

### DNA Extraction from Slide Section

Volumes are given for extraction of one section. Regions of tumour identified by histopathology on one section were isolated from adjacent sections by scraping relevant area from slide into an eppendorf tube. The material from a 20 micron section was resuspended in 100 μl 0.5% Tween-20 (Sigma Aldrich), heated to 90° C. for 10 minutes then cooled to 55° C. Proteinase K (2 μl, 10 mg/ml) was added to the suspension, the solution was mixed and incubated at 55° C. for 3 hours with occasional mixing. Chelex-100™ (C7901, Sigma) (100 μl, 5% (w/v) in Tris EDTA) was added and the suspension was incubated at 99° C. for 10 minutes. The extracted DNA was recovered by centrifugation at 10500×g for 15 minutes, the solution below the wax layer which formed was transferred to a clean tube. The solution was heated to 45° C. before adding chloroform (100 μl). The suspension was mixed before further centrifugation at 10500×g for 15 minutes, DNA'was then recovered from the upper aqueous layer by ethanol precipitation. The DNA pellet was rinsed in 70% ethanol, recovered by a pulse of centrifugation, air dried and dissolved in water (50 μl).

### Example 3

### Amplification Refractory Mutation System for Detection of Met918Thr Mutation in RET Using ARMS Primers

An Amplification Refractory Mutation System assay (ARMS) may be used to detect the presence of a nucleotide base change in the RET gene compared to a background of normal DNA. Each ARMS assay is specific for a given mutation e.g. designed to detect a change from one base to another base at a given position. The assay is multiplexed with a second PCR reaction that detects the presence of DNA in the reaction, thereby indicating successful PCR. TaqMan™ technology is used to detect the PCR products of both reactions using TaqMan™ probes labelled with different fluorescent tags.

PCR was performed on 5 μl of genomic DNA containing varying proportions of mutant and wild type DNA and varying concentrations of input DNA. A total reaction volume of 25 μl was used for each PCR. 1 Unit of Amplitaq gold DNA polymerase (N80080246, ABI) was used in each reaction with final concentrations of 3.5 mM magnesium chloride, 200 μM dNTPs (deoxyribonucleotide triphosphates) and 1.0 μM of each ARMs mutant forward primer and ARMS reverse primer short (see Table 1) in buffer (final buffer composition 15 mM Tris-HCl Ph 8.3, 50 mM KCl). TaqMan™ probes (Eurogentech) were added to each reaction at a final concentration of 0.5 μM. Cycle conditions were as follows: 95° C. for 10 minutes followed by 40 cycles of 94° C. for 45 seconds, 60° C. for 45 seconds, 72° C. for 1 minute in a Real Time PCR instrument (e.g. Stratagene Mx4000 or ABI 7900). 10 copies of mutant could be detected in a background of 1000 copies of wild type DNA.

### Example 4

### Amplification Refractory Mutation System for Detection of Met918Thr Mutation in RET from Clinical Samples Using ARMS LNA Primers

Formalin fixed tissue samples were obtained from patients participating in a clinical trial to assess the activity of vandetanib in sporadic medullary thyroid cancer.

DNA was extracted as described in Example 2. Nineteen samples were available for analysis, including two samples from a single patient. All samples were analysed in triplicate and scored by two independent operators.

An Amplification Refractory Mutation System assay (ARMS) was used to detect the presence of a nucleotide base change in the RET gene compared to a background of normal DNA. The assay was multiplexed with a second PCR reaction that detects the presence of DNA in the reaction, thereby indicating successful PCR. TaqMan™ technology was used to detect the PCR products of both reactions using TaqMan™ probes labelled with different fluorescent tags.

PCR was performed on genomic DNA containing varying proportions of mutant and wild type DNA and varying concentrations of input DNA. A total reaction volume of 25 μl was used for each PCR. 1 Unit of Amplitaq gold DNA polymerase was used in each reaction with final concentrations of 3.5 mM magnesium chloride, 200 μM dNTPs and 1.0 μM of each ARMS LNA Mutant Forward primer and ARMS Reverse Primer Short (see Table 1) in buffer (final buffer composition 15 mM Tris-HCl Ph 8.3, 50 mM KCl). TaqMan™ probes were added to each reaction at a final concentration of 0.5 μM. Cycle conditions were as follows: 95° C. for 10 minutes followed by 40 cycles of 94° C. for 45 seconds, 60° C. for 1 minute, 72° C. for 45 seconds in a Real Time PCR instrument (e.g. Stratagene Mx4000 or ABI 7900).

Six samples could not be scored because of low DNA concentrations. Mutations were detected in 8/13 of the evaluable samples, giving a mutation frequency of 61.5%. Sequencing was performed on all samples to confirm mutation status; but sequence data could only be obtained from samples with DNA concentrations>50 copies and thus only 4 samples gave readable sequence at codon 918. The results obtained by sequencing were fully concordant with the data from the ARMS assay.

TABLE 2
Mutation Detection in Clinical Samples

| | DNA | | |
|---|---|---|---|
| Sample Identifier | copies | Mutation status | Sequence Confirmation |
| E2802001 | <5 | Assay fail | |
| E1701004 | <5 | Assay fail | |
| E1701003 | <5 | Assay fail | |
| E1001001 | <5 | Assay fail | |
| E0002001-a | <5 | Assay fail | |
| E0002004 | <5 | Assay fail | |
| E2801001 | <5 | Mutant | |
| E0002005 | 170 | Mutant | Yes |
| E2002002 | 240 | Mutant | Yes |
| E2002001 | <5 | Mutant | |
| E1001002 | <5 | Mutant | |
| E1001004 | <5 | Mutant | |
| E0002001-b | <5 | Mutant | |
| E0002002 | 430 | Mutant | |
| E0011003 | 410 | Wild-type | Yes |
| E1707002 | 120 | Wild-type | Yes |
| E3001001 | 90 | Wild-type | |
| E1201002 | <5 | Wild-type | |
| E0002003 | 10 | Wild-type | |

Two samples (-a, -b) were obtained from patient E0002001

### Example 5

### Comparison of Specificity of Conventional DNA Primers Compared to LNA Primers in an ARMS Assay to Detect the M918T RET Mutation

An experiment was performed to determine the threshold at which ARMS primers designed to detect the M918T RET mutation can generate a signal from wild type DNA. PCR was performed on 5 μl of either mutant or wild type genomic DNA representing different concentrations of input DNA. A total reaction volume of 25 μl was used for each PCR. 1 Unit of Amplitaq gold DNA polymerase was used in each reaction with final concentrations of 3.5 mM magnesium chloride, 200 μM dNTPs and 1.0 μM of each ARMs mutant forward primer and ARMS reverse primer short (see Table 1) in buffer (final buffer composition 15 mM Tris-HCl Ph 8.3, 50 mM KCl). TaqMan™ probes were added to each reaction at a final concentration of 0.5 μM. Cycle conditions were as follows: 95° C. for 10 minutes followed for up to 45 cycles of 94° C. for 45 seconds, 60° C. for 45 seconds, 72° C. for 1 minute in a Real Time PCR instrument (e.g. Stratagene Mx4000 or ABI 7900).

A signal is generated from 1000 copies of mutant DNA at 29 cycles using the conventional DNA ARMS primers (FIG. 1). However, a signal is also generated from a sample containing either 1000 or 10000 copies of wild type DNA. Although the signal from wild type DNA is generated at 35 and 32 cycles respectively, the potential to obtain a signal from wild type DNA could limit the use of conventional DNA ARMS primers in the clinical setting where it is common for fixed tumour samples to contain mixtures of normal and tumour tissue.

A similar experiment was performed using LNA modified primers. In this experiment, a signal is generated from the sample containing 1000 copies of mutant DNA at 31 cycles but no signal is generated from the samples containing either 1000 or 10000 copies of wild type DNA even when the analysis is extended to 45 cycles (FIG. 2).

### Example 6

### Selection of Patients for Treatment

Detection of a mutation in the RET gene in a tumour sample can be used to improve the selection of a patient who is a candidate for treatment with vandetanib or other inhibitors of the RET tyrosine kinase, either as monotherapy or in combination therapy, whereby to predict an increased likelihood of response to vandetanib or other inhibitors of the RET tyrosine kinase.

1. A method for predicting the likelihood that a patient who is a candidate for treatment with a RET drug will respond to said treatment, comprising determining whether the sequence of RET in a sample obtained from the patient
at position 105, as defined in SEQ ID NO:1, is not thymine; or
at position 918, as defined in SEQ ID NO:2, is not methionine,
whereby to predict an increased likelihood of response to the RET drug.

2. A method according to claim 1 wherein
position 105 is cytosine; or
position 918 is threonine.

3. A method according to claim 1 wherein position 105 is cytosine.

4. Use of a method according to claims 1 to 3 to assess the pharmacogenetics of a RET drug.

5. A method of treating a patient who is a candidate for treatment with a RET drug, comprising:
(i) determining whether the sequence of RET in a sample obtained from the patient at position 105, as defined in SEQ ID NO: 1, is not thymine; or
(ii) determining whether the sequence of RET in a sample obtained from the patient at position 918, as defined in SEQ ID NO: 2, is not methionine,
and administering an effective amount of the RET drug.

6. A method according to claim 5 wherein
position 105 is cytosine; or
position 918 is threonine,
and administering an effective amount of the RET drug.

7. A method according to claim 5 wherein position 105 is cytosine.

8. A method according to claim 3 wherein the method for determining the sequence of RET in a sample obtained from a patient is selected from any one of amplification refractory mutation system, restriction fragment length polymorphism or WAVE analysis.

9. A method according to claim 8 wherein the method for determining the sequence of RET in a sample obtained from a patient is the amplification refractory mutation system.

10. A method according to claim 3, 7, 8 or 9 comprising using an ARMS mutant forward primer capable of recognising the sequence of RET at position 105, as defined in SEQ ID NO: 1.

11. A method according to claim 3, 7, 8, or 9 comprising using an ARMS mutant forward primer and an ARMS reverse primer optimized to amplify the region of a RET sequence comprising position 105, as defined in SEQ ID NO: 1.

12. A method according to claim 10, wherein the ARMS mutant forward primer comprises SEQ ID NO:9.

13. A method according to claim 1 or 5 wherein the RET drug is a RET tyrosine kinase inhibitor.

14. A method according to claim 13 wherein the RET drug is vandetanib.

15. A method according to claim 13 wherein the RET drug is cediranib.

16. An ARMS mutant forward primer capable of recognising the sequence of RET at position 105, as defined in SEQ ID NO: 1.

17. An ARMS mutant forward primer according to claim 16, comprising SEQ ID NO:9.

18. A diagnostic kit comprising an ARMS mutant forward primer of claim 16 or 17.


SEQUENCE LISTING (11 sequences)

SEQ ID NO: 1 - DNA, 320 bp, Homo Sapiens
gatagggcctggccttctcctttacccctccttcctagagagttagagtaacttcaatgtctttattccatcttctctttagggtcggattccagttaaatggatggcaattgaatccctttttgatcatatctacaccacgcaaagtgatgtgtaagtgtgggtgttgctctcttggggtggaggttacagaaacacccttatacatgtagtggggccacgacgcccgtctgtgcagcttggccagggaattgcactggccctgagcacctgtctgcagtgctagccctctgcaatgaccccctcactgaggggccttc

SEQ ID NO: 2 - protein, 1114 aa, Homo Sapiens
MAKATSGAAGLRLLLLLLLPLLGKVALGLYFSRDAYWEKLYVDQAAGTPLLYVHALRDAPEEVPSFRLGQHLYGTYRTRLHENNWICIQEDTGLLYLNRSLDHSSWEKLSVRNRGFPLLTVYLKVFLSPTSLREGECQWPGCARVYFSFFNTSFPACSSLKPRELCFPETRPSFRIRENRPPGTFHQFRLLPVQFLCPNISVAYRLLEGEGLPFRCAPDSLEVSTRWALDREQREKYELVAVCTVHAGAREEVVMVPFPVTVYDEDDSAPTFPAGVDTASAVVEFKRKEDTVVATLRVFDADVVPASGELVRRYTSTLLPGDTWAQQTFRVEHWPNETSVQANGSFVRATVHDYRLVLNRNLSISENRTMQLAVLVNDSDFQGPGAGVLLLHFNVSVLPVSLHLPSTYSLSVSRRARRFAQIGKVCVENCQAFSGINVQYKLHSSGANCSTLGVVTSAEDTSGILFVNDTKALRRPKCAELHYMVVATDQQTSRQAQAQLLVTVEGSYVAEEAGCPLSCAVSKRRLECEECGGLGSPTGRCEWRQGDGKGITRNFSTCSPSTKTCPDGHCDVVETQDINICPQDCLRGSIVGGHEPGEPRGIKAGYGTCNCFPEEEKCFCEPEDIQDPLCDELCRTVIAAAVLFSFIVSVLLSAFCIHCYHKFAHKPPISSAEMTFRRPAQAFPVSYSSSGARRPSLDSMENQVSVDAFKILEDPKWEFPRKNLVLGKTLGEGEFGKVVKATAFHLKGRAGYTTVAVKMLKENASPSELRDLLSEFNVLKQVNHPHVIKLYGACSQDGPLLLIVEYAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGMSDPNWPGESPVPLTRADGTNTGFPRYPNDSVYANWMLSPSAAKLMDTFDS

SEQ ID NO: 3 - DNA, 29 bp, Artificial Sequence
  PCR Primer
ctttagtgtcggattccagttaaatggtc

SEQ ID NO: 4 - DNA, 22 bp, Artificial Sequence
  22; PCR Primer
tgcaattccctggccaagctgc

SEQ ID NO: 5 - DNA, 40 bp, Artificial Sequence
  PCR Primer
ctacaccacgcaaagtgatgtgtaagtgtgggtgttgctc

SEQ ID NO: 6 - DNA, 22 bp, Artificial Sequence
  PCR PRIMER
aggacaccgaggaagaggactt

SEQ ID NO: 7 - DNA, 23 bp, Artificial Sequence
  PCR PRIMER
ggaatcaccttctgtcttcattt

SEQ ID NO: 8 - DNA, 29 bp, Artificial Sequence
  PCR PRIMER
ccatcttcttcctgcctgatgaggggaaa

SEQ ID NO: 9 - DNA, 21 bp, Artificial Sequence
  PCR PRIMER
tcggattccagttaaatggtc

SEQ ID NO: 10 - DNA, 25 bp, Artificial Sequence
  PCR Primer
tgatgtgtaagtgtgggtgttgctc

SEQ ID NO: 11 - DNA, 17 bp, Artificial Sequence
  PCR PRIMER
ctgcctgatgaggggaa